(19) United States
(12) Patent Application Publication
(10) Pub. No.: US 2022/0340615 A1
(43) Pub. Date: Oct. 27, 2022

(54) PROCESS FOR THE PURIFICATION OF PHYTOSTEROL

(71) Applicants: BASF SE, Ludwigshafen am Rhein (DE); BASF HEALTH AND CARE PRODUCTS FRANCE S.A.S., Levallois-Perret (FR)

(72) Inventors: Valerie EYCHENNE, Boussens (FR); Yannick BASSO, Boussens (FR); Olivier VALLEJO, Boussens (FR)

(21) Appl. No.: 17/762,752

(22) PCT Filed: Sep. 24, 2020
(86) PCT No.: PCT/EP2020/076727

§ 371 Date: Mar. 23, 2022

(30) Foreign Application Priority Data
Sep. 27, 2019 (EP) 19200108.9

(51) Int. Cl. C07J 9/00; B01D 9/00

(52) U.S. Cl. CPC C07J 9/00; B01D 9/0004; B01D 9/0045; B01D 2009/0086

(57) ABSTRACT

A process for purification of phytosterols, said process comprising, a. providing a liquid mixture comprising a phytosterol, lower alcohol, wherein the lower alcohol is present in an amount of from 25 to 800% by weight, based on the amount of phytosterol; b. cooling the mixture to form phytosterol crystals, wherein the crystals are formed at a temperature of from of from 10° C. to 75° C., preferably 15° C. to 50° C., more preferably 20° C. to 45° C., even more preferably 25° C. to 35° C. c. separating the phytosterol crystals from the remainder of the mixture by filtration; d. subjecting the phytosterol crystals to washing with a solvent system comprising at least one polar aprotic solvent to obtain purified phytosterol; e. optionally repeating step (d); f. drying the washed phytosterol crystals; g. optionally melting and drying in molted state to remove traces of remaining solvent; and h. optionally subjecting to a particle-forming process to obtain solid sterol particles.

### FIELD OF INVENTION

The presently claimed invention relates to phytosterols for the use in food additives, and more particularly, to a new, simplified process for the purification of phytosterols which are suitable for the use as food additives.

### BACKGROUND

Phytosterols and their esters possess hypocholesterolaemic properties, i.e. these substances are capable of lowering the cholesterol level in the blood. Accordingly, they are used as food additives, for example for the production of margarine, frying oils, sausages, ice creams and the like. The production of sterols and other unsaponifiable constituents, such as tocopherols for example, from distillates obtained in the deacidification of vegetable oils has already been variously described in the patent literature, cf. EP-A20 610 742 (Hoffmann-LaRoche), GB-A1 2,145,079 (Nisshin Oil Mills Japan) and EP-A1 0 333 472 (Palm Oil Research and Development Board).

EP 0 656 894 B1 (Henkel) describes a process for the production of sterols in which a residue from the distillation of methyl esters consisting essentially of glycerides, sterols, sterol esters and tocopherols is transesterified with methanol in the presence of alkaline catalysts. After 25 neutralization of the catalyst, removal of the excess methanol by distillation and, optionally, removal of the catalyst by washing, the sterols are crystallized by lowering the reaction temperature from about 65 to 20° C. The thus obtained crystals are washed with methanol and water. Unfortunately, the yield of sterols is unsatisfactory.

EP 2635 592 B1 (Verbio) discloses a method for obtaining phytosterols and tocopherols using multi-phase separation systems to isolate the sterol and/or tocopherol.

EP1179535 B1 and EP1179536 B1 (both: BASF) disclose processes for the production of sterols using a two-step-transesterification to obtain sterols from vegetable oil distillates. Crystallization of the obtained sterols and washing with methanol and fatty acid methyl ester (hereinafter as “FME”, but generally known also as “FAME”) is disclosed as subsequent process steps in dependent claims. Although in EP1179536 B1 “methyl ester” is disclosed as employed solvent in the examples, the “methyl ester” actually used in the examples and disclosed in the description is the FME from the transesterifications of the vegetable oil. EP1179535 B1 discloses in its examples the use of “FME”; in [0036] and [0042] EP1179535 B1 also discloses that the crystals obtained in examples a) and b) “are washed with suitable solvents”. However, which solvents those actually might be is not disclosed. EP1169335 B1 (BASF) discloses a process for the crystallization of sterols from a specific mixture of methanol and FME in certain ratios and washing of the obtained crystals. Objective of this disclosure is to provide sterols in high yields and “good color quality”. Key is according to this disclosure the optimum amount and ratio of methanol during crystallization and thus the crystallization temperature which is said to lead to the desired improvement. The obtained crystals are then washed with FME, which step is said to further improve the color quality of the sterol crystals obtained. It is to be noted that the 50 “methylester” disclosed by EP1169335 B1 clearly is the “fatty acid ethyl ester”, as both descriptions/terms are used inter-changingly as can be seen from e.g. [0008], which mentions twice the washing of the crystals but uses “methyl ester” at the first occasion and “FME” on the second occasion. Claim 1 in the binding German version thus correctly uses the term “FME” (whereas claim 1 in the English translation using incorrectly the term “fatty acid 55 ester”).

Several methods are reported to improve the yield of sterol. However, improving the color of the sterol while maintaining other advantageous properties of the purification process such as improved yield and reduced impurities in the sterol product remains a challenge

### SUMMARY OF THE INVENTION

It has surprisingly been found that the color and purity of the phytosterol is significantly influenced by the solvent that is used for purification Thus, the choice of solvent that is used for the purification process plays a significant role in improving the color of the phytosterol and reducing the amount of impurities without compromising on the yield of the final product.

Hence in one aspect, the presently claimed invention relates to a purification process, wherein the color of the final phytosterol product is significantly improved and the amount of phytosterol ester as an impurity is significantly reduced by selecting at least one polar aprotic solvent for washing the final phytosterol product.

It also has surprisingly been found that the presently claimed invention leads to a significantly reduced solvent content in the final phytosterol product.

### DESCRIPTION OF THE INVENTION

Although the presently claimed invention will be described with respect to particular embodiments, this description is not to be construed in a limiting sense.

Before describing in detail exemplary embodiments of the presently claimed invention, definitions important for understanding the presently claimed invention are given. As used 25 in this specification and in the appended claims, the singular forms of “a” and “an” also include the respective plurals unless the context clearly dictates otherwise. In the context of the presently claimed invention, the terms “about” and “approximately” denote an interval of accuracy that a person skilled in the art will understand to still ensure the technical effect of the feature in question. The term typically indicates a deviation from the indicated numerical 30 value of ±20%, preferably ±15%, more preferably ±10%, and even more preferably ±5%. It is to be understood that the term “comprising” is not limiting. For the purposes of the presently claimed invention the term “consisting of” is considered to be a preferred embodiment of the term “comprising of”. If hereinafter a group is defined to comprise at least a certain number of embodiments, this is meant to also encompass a group which preferably 35 consists of these embodiments only.

In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, 40 minutes, hours, days, weeks, months or even years between such steps, unless otherwise indicated in the application as set forth herein above or below. Preferably, however, the steps are performed in the numerical or hierarchical order implied by it, i.e. at first a, then b, then c etc., first i), then ii), then iii) etc. It is to be understood that this invention is not limited to the particular methodology, protocols, reagents etc. described herein as these may vary, provided that the intention and objective of this invention is or may be achieved with those. It is also to be understood that the terminology used herein is for the purpose of describing particular embodiments only and is not intended to limit the scope of the presently claimed invention that will be limited only by the appended claims. Unless defined otherwise, all technical and scientific terms used herein have the same meanings as commonly understood by one of ordinary skill in the art.

Unless otherwise indicated, the following definitions are set forth to illustrate and define the meaning and scope of the various terms used to describe the invention herein and the appended claims. These definitions should not be interpreted in the literal sense as they are 55 not intended to be general definitions and are relevant only for this application

The term “final sterol product” signifies the phytosterol which is obtained after the purification steps.

The term “(oil) distillate” encompasses edible vegetable oil distillates (VODs) which are even preferred.

The term “(oil) distillation residue” encompasses transesterified oil distillation residues which are even preferred. Said transesterified oil distillation residues are preferably fatty acid alkyl ester distillation residues, more preferably fatty acid methyl ester distillation residues in particular from the production of bio-diesel.

The term “partial glycerides” encompasses all combinations of mono-, di- and/or triglycerides. In case of oil distillates as starting material, there are only or nearly only triglycerides and no mono- and diglycerides, whereas, in case of typical oil distillation residues, there are mainly triglycerides and diglycerides and only a few monoglycerides. Meaning of the terms that are not defined herein are generally known to a person skilled in the art or in the literature.

It is also intended that of course the various embodiments and preferred options of the various process steps disclosed herein are to be combined within the actual complete process, so that for a specific performance of this overall process for one process step the general outline is selected, for another process step within this overall process the preferred embodiment and for the again another process step the most preferred option etc.

In an embodiment, the presently claimed invention relates to a process for purification of phytosterols, said process comprises at least the steps of:
a. providing a liquid mixture comprising a phytosterol and a lower alcohol, wherein the lower alcohol is present in an amount of from 25 to 800% by weight, based on the amount of phytosterol;
b. cooling the liquid mixture of step a. to form phytosterol crystals, wherein the phytosterol crystals are formed at a temperature of from 10° C. to 75° C., preferably 15° C. to 50° C., more preferably 20° C. to 45° C., even more preferably 25° C. to 35° C.;
c. separating the phytosterol crystals of step b. from the remainder of the liquid mixture by mechanical means such as filtration and/or centrifugation;
d. subjecting the phytosterol crystals of step c. to washing with a solvent system comprising at least one polar aprotic solvent to obtain purified phytosterol crystals;
e. optionally repeating step (d);
f. drying the purified phytosterol crystals.

In an embodiment, the presently claimed invention relates to a purification process, wherein the color of the final sterol product is significantly improved and/or the amount of sterol ester as an impurity is significantly reduced.

In an embodiment, the presently claimed invention relates to a significantly reduced solvent content in the final sterol product.

In an embodiment, the presently claimed invention relates to the recovery of phytosterol from the residues of the deacidification of vegetable oils and its subsequent purification to obtain pure phytosterol.

In an embodiment, the presently claimed invention relates to the recovery of phytosterol from the prior art process as disclosed in any of EP1169335 B1, EP1169336 B1 or EP 2 635 592 B1, in which either i) the sterol crystals obtained by said processes are then subjected to the process of the present invention or ii) the sterol containing solutions/dispersions of said processes are adjusted as such that they comply with the requirements of the process step a) as defined:
a) providing a liquid mixture comprising a phytosterol and a lower alcohol, wherein 55 the lower alcohol is present in an amount of from 25 to 800% by weight, based on the amount of phytosterol;
   and then following the further process steps, said process comprising at least the steps of:
b) cooling the liquid mixture of step a. to form phytosterol crystals, wherein the phytosterol crystals are formed at a temperature of from 10° C. to 75° C., preferably 15° C. to 50° C., more preferably 20° C. to 45° C., even more preferably 25° C. to 35° C.;
c) separating the phytosterol crystals of step b. from the remainder of the liquid mixture by mechanical means such as filtration and/or centrifugation;
d) subjecting the phytosterol crystals of step c. to washing with a solvent system comprising at least one polar aprotic solvent to obtain purified phytosterol crystals;
e) optionally repeating step (d);
f) drying the purified phytosterol crystals;
g) optionally melting and drying in molted state to remove traces of remaining solvent;
h) optionally subjecting to a particle-forming process to obtain solid sterol particles

Transesterification

In an embodiment of the presently claimed invention, the process of transesterification of vegetable oil methyl ester distillation residues, and the work up can be carried out as described in EP 0 656 894 B1. Suitable starting materials are the distillation residues obtained from vegetable oil-based processes, for example, in the production of FMEs (also known as “bio-diesel”), for example, in the production of FMEs based on rapeseed oil, but could be also performed using sterol-containing products obtained from processes based on tall oil as starting material such as the recovery of sterols from tall oil pitch, such as pitch obtained from e.g. birch bark; specifically preferable starting materials are distillation residues obtained from vegetable oil treatments and distillations such as, for example, obtained in the production of FMEs (also known as “bio-diesel”). Further when the process relates to the production of the sterol fractions, reference is made to EP 0 656 894 B1. The process is particularly suitable for the production of sterols based on vegetable oils, particularly the residues from the distillation of vegetable oil methyl ester.

In an embodiment of the presently claimed invention, residues from the distillation of transesterified, more particularly non-refined oils preferably with a residual acid value below 2, are used as raw materials for the production of sterols.

Such residues may be obtained as outlined herein above, using the known prior art processes. Especially suitable residues are such from the work-up of vegetable oils containing sterols and usually also tocopherols. These residues are obtained by several esterifications and transesterifications, treatment with acid etc., all of which is known in the art. One such known process is the process to produce bio-diesel, i.e. fatty acid methyl ester (FME).

Preferably, the oil distillation residue comprises a residue derived from an oil selected from the group consisting of soybean oil, sunflower oil, rapeseed oil, high erucic acid rapeseed oil (HEAR), low eruric acid rapeseed oil (CANOLA; CANadian Oil Low eruic Acid), coconut oil, palm oil, palm kernel oil, and mixtures thereof, more preferably, the oil distillation residue comprises a residue derived from soybean oil, sunflower oil, rapeseed oil such as HEAR or CANOLA, even more preferably, the oil distillation residue comprises a residue derived from sunflower oil, rapeseed oil, preferably HEAR.

These residues are preferably residues from coconut oil, from palm kernel oil, from palm oil, from soybean oil, from sunflower oil, from rapeseed oil such as from HEAR and/or 50 CANOLA, more preferably from soybean oil, sunflower oil, rapeseed oil such as HEAR, even more preferably from sunflower oil and/or rapeseed oil, and especially HEAR, with acid values of 0 to 10, preferably from 0 to 6 and contain mixtures of di- and triglycerides, FMEs, sterol esters, wax esters and free sterols, preferably 1 to 7% by weight triglycerides, 3 to 15% by weight diglycerides, 15 to 40% by weight FMEs, 40 to 50% by weight, in particular 42 to 47% by weight sterol esters, 3 to 4% by weight wax esters and 3 to 15% by weight free sterols and small quantities of monoglycerides.

In another embodiment of the presently claimed invention, oil distillates are used as raw materials for the production of sterols. These distillates are preferably such of coconut oil, of palm kernel oil, of palm oil, of soybean oil, of sunflower oil, of rapeseed oil such as from HEAR and/or CANOLA, more preferably of soybean oil, sunflower oil, rapeseed oil such as from HEAR, even more preferably of sunflower oil and/or rapeseed oil from HEAR, containing 45 to 65% by weight triglycerides and 35 to 55% by weight sterol esters summing up to 100%.

Thus, in a preferred embodiment of the presently claimed invention, the phytosterol-rich fractions from the transesterification and distillations of rapeseed oil (“rapeseed sterols”) or soybean oil (“soy sterols”), sunflower oil are used as starting material.

The processes disclosed in EP1179535B1, EP1179536B1 and EP 2 635 592 B1, can also be used and modified using this present invention.

Thus, in three further embodiments, the process as carried out in EP1179535B1, EP1179536B1 or EP 2 635 592 B1 can be implemented for the transesterification reactions to yield sterols, then to be followed by the presently disclosed process of crystallization and specifically the process of purification.

After crystallization and purification as outlined hereinafter in detail, a typical further work-up procedure can be employed: the washed sterol crystals can be dried using conventional dryers of all kinds, to remove remaining solvents. Application of reduced pressure helps to increase the removal of solvent traces. Following such drying, the crystals can be melted preferably again under reduced pressure to also remove solvent traces enclosed within the crystals. The melted sterols can then be formed to particles by typical known procedures, such as prilling (i.e. forming droplets close to spherical shapes) or simply in dripping towers, in which molten material is dropped into colder air or gases, all such methods to finally obtained solid, particulate sterols, which are preferably forms that do not show dusting but good flowability and preferably a high density, to obtain sterols particulates with easy handling properties.

Crystallization

In another embodiment, the purification of the sterol fractions which, apart from the lower alcohol, mainly contain methyl esters (specifically when those residues stem from the 35 transesterification and distillation of vegetable oils such as the so-called bio-diesel processes), takes place in a known manner, i.e. the hot mixtures (ca. 50-65° C. when working at atmospheric pressure, accordingly higher temperatures when working at elevated pressures and. usually not preferable—lower temperatures when working at reduced pressures; all following temperatures and temperatures ranges are given for atmospheric pressure and 40 thus and increased or lowered pressure higher or lower temperatures apply as to be readily acknowledged by a person of ordinary skill) are slowly cooled to form the phytosterol crystals, which are formed at a temperature of from 15° C. to 50° C., preferably 20° C. to 45° C., more preferably 25° C. to 35° C., even more preferably 20° C. to 30° C., in a crystallizer. If necessary, the alkaline catalysts that are present in the mixture from the transesterification 45 can be neutralized beforehand, for example by addition of citric acid or other suitable organic or inorganic acids that are also suitable or acceptable for the intended use of the sterols later; preferably, such neutralization is omitted, if the feed for the crystallization allows for.

In an embodiment of the presently claimed invention, the lower alcohol is selected from the 50 group consisting of methanol, ethanol and isopropyl alcohol. Preferably the lower alcohol is methanol.

In an embodiment of the presently claimed invention, only those mixtures which already have a ratio by weight of sterol to methanol of 100:25 to 100:75 from their production should 55 be used for starting the crystallization procedure and further process steps of the presently claimed invention. Otherwise methanol has to be added or distilled off to adjust to the required ratios, such adjustment being easily accomplished by standard methods and equipment. Under these conditions, and for the temperatures given afterwards at atmospheric pressure—for higher or lower pressure higher or lower temperatures apply as outlined before -, the crystallization begins at temperatures of 60-65° C., but could be also done at higher temperatures, if the crystallization is done at elevated pressures and/or if the solvent mixture employed has a boiling point higher than the one disclosed as preferred herein.

In an embodiment of the presently claimed invention, the ratio of sterol:methanol is in the range of 1:0.1 to 1:5, preferably 1:0.5 to 1:3, more preferably 1:0.5 to 1:2.5.

In an embodiment of the presently claimed invention, the sterol containing phase, which primarily contains sterol crystals, may subsequently be washed with methanol, wherein the quantity of methanol is in the range from 20% to 800%, preferably in the range from 125% to 600%, more preferably in the range from 200% to 400% in each case based on the mass of the sterol crystal phase.

In an embodiment of the presently claimed invention, the sterol containing phase, which primarily contains sterol crystals, may subsequently be washed with methanol, wherein the quantity of methanol is in the range from 20% to 100%, preferably in the range from 25% to 90%, more preferably in the range from 25% to 75%, more preferably in the range of 25% to 60%, even more preferably in the range of 30% to 50% in each case based on the mass of the sterol crystal phase. This embodiment is preferred over the embodiment using larger amounts of solvents for this step as it is more economical in solvent usage.

In an embodiment of the presently claimed invention, the phytosterol crystals are formed at a temperature of from 10° C. to 75° C., preferably 15° C. to 50° C., more preferably 20° C. to 45° C., even more preferably 25° C. to 35° C. even more preferably 20° C. to 30° C., such as 20° C., 21° C., 22° C., 23° C., 24° C., 25° C., 26° C., 27° C., 28° C., 29° C., 30° C., 31° C., 32° C., 33° C., 34° C. or 35° C.

Purification of the Phytosterol

In yet another embodiment of the presently claimed invention, the sterol crystals are separated by mechanical means such as filtration and/or centrifugation, preferably filtration.

In a further embodiment of the presently claimed invention, the separated sterol crystals are subjected to further purification steps.

In yet another embodiment of the presently claimed invention, the sterol crystals are further purified using a solvent system.

In yet another embodiment of the presently claimed invention, the purification of the sterol 40 crystals occurs in the presence of a solvent system which comprises at least one polar aprotic solvent.

In a further embodiment of the presently claimed invention, the purification of the sterol fraction occurs in the presence of at least one polar aprotic solvent which is selected from the 45 group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, with ethyl acetate, acetone, methyl ethyl ketone, methyl acetate, heptane and hexane being preferred, and with ethyl acetate, methyl ethyl ketone and methyl acetate being even more preferred.

In yet another embodiment of the presently claimed invention, the purification of the sterol fraction occurs in the presence of at least one polar aprotic solvent which is selected from the group consisting of ethyl acetate, acetone, methyl ethyl ketone and methyl acetate, even more preferred from ethyl acetate, methyl ethyl ketone and/or methyl acetate.

In a particularly preferred embodiment, methyl acetate is used as the sole polar aprotic solvent.

In a further embodiment of the presently claimed invention, the purification of the sterol fraction occurs in the presence of at least one polar aprotic solvent and at least one polar protic solvent which are mixed together and/or form an azeotrope in the solvent system.

In an embodiment of the presently claimed invention, the at least one polar protic solvent is selected from the group of water, ethanol, methanol, isopropyl alcohol, butanol and acetic acid.

In yet another embodiment of the presently claimed invention, the at least one polar protic solvent is from water, ethanol, methanol and/or isopropyl alcohol.

In a particularly preferred embodiment methanol is used as the sole polar protic solvent, which may—but not preferred—contain however small amounts of water

In an embodiment of the presently claimed invention, the polar aprotic solvent is present in the range of 25 to 75% by weight, based on the amount of phytosterol, preferably in the range of 30% to 50% by weight, based on the amount of phytosterol, and every value in between 30% to 50%, based on the amount of phytosterol.

In an embodiment of the presently claimed invention, the polar protic solvent is present in the range of 5 to 50% by weight, based on the amount of phytosterol, preferably in the range of 10 to 30% by weight, based on the amount of phytosterol, and every value in between 10% to 30%, based on the amount of phytosterol.

In an embodiment of the presently claimed invention, a process for purification of phytosterols comprises at least the steps of,
a. providing a liquid mixture comprising a phytosterol and a lower alcohol, wherein the lower alcohol is present in an amount of from 25 to 75% by weight, based on the amount of phytosterol;
b. cooling the liquid mixture of step a. to form phytosterol crystals, wherein the phytosterol crystals are formed at a temperature of from 10° C. to 75° C., preferably 15° C. to 50° C., more preferably 20° C. to 45° C., even more preferably 25° C. to 35° C.;
c. separating the phytosterol crystals of step b. from the remainder of the liquid mixture by mechanical means such as filtration and/or centrifugation;
d. washing the phytosterols crystals of step c. with a solvent system comprising at least one polar aprotic solvent and at least one polar protic solvent to obtain purified phytosterol crystals;
e. optionally repeating step d. at least once, more preferably at least 2 times, even more preferably at least 3 times;
f. optionally repeating steps a. to e. by re-dissolving the phytosterols in a liquid mixture according to step a. and then repeating steps b. to f. and then continue with the following step g;
g. drying the purified phytosterol crystals obtained from the preceding step,
h. optionally melting and drying in molted state to remove traces of remaining solvent;
i. optionally subjecting to a particle-forming process to obtain solid sterol particles;

with the at least one polar aprotic solvent being selected from the group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, with ethyl acetate, acetone, methyl ethyl ketone, methyl acetate, heptane and hexane 50 being preferred, and with ethyl acetate, methyl ethyl ketone and methyl acetate being even more preferred, and

with the at least one polar aprotic solvent being selected from the group of water, ethanol, methanol, isopropyl alcohol, butanol and acetic acid, with water, ethanol, methanol and/or isopropyl alcohol being more preferred and methanol being most preferred, even more 55 preferably methanol as sole polar aprotic solvent.

In a further embodiment of the presently claimed invention, the sterol crystals could be washed with methyl ester, such as methyl acetate, after washing with at least one polar aprotic solvent and at least one polar protic solvent.

In a further embodiment of the presently claimed invention, the sterol crystals could be washed with methyl ester, such as methyl ethyl ester and/or methyl fatty acid ester, after step c and before step d to “pre-wash” the crystals further to further increase the purity and/or color of the to be obtained sterol crystals.

The washed sterol crystals can be dried using conventional dryers of all kinds, to remove remaining solvents. Application of reduced pressure helps to increase the removal of solvent traces. This step serves a drying or “pre-drying”, depending on the method employed and the desired content of residual solvent in the final sterol product to be obtained. The latter of course mainly depends on the intended use of the sterols.

Following the “conventional” drying of the previous step (g), the (pre-)dried crystals can be melted preferably under reduced pressure to remove solvent traces enclosed within the crystals. By this, the residual content of solvents can be lowered even more so as to achieve certain higher product qualities being usable also for critical applications, e.g. direct applications to human beings in nutritional or pharmaceutical products.

The melted sterols need to be solidified. That could be done either by simple cooling with stirring of any kind, e.g. in an extruder, a paddle dryer and the like. Other known methods for solidification of melts are prilling, in apparatuses such as prillers including jet-prillers, which can form droplets close to spherical shapes, or simply in dripping towers, in which molten material is dropped into colder air or gases, all such methods to finally obtained solid, particulate sterols, which are preferably in forms that do not show dusting but good flowability and preferably a high density, to obtain sterols particulates with easy handling properties.

Thus, in a further embodiment, the sterols obtained—and preferably (pre-)dried—are subjected to a particle forming process, preferably to prilling, more preferably to jet-prilling, which is preferably done under liquid nitrogen, to obtain solid, close to spherical, low to non-dusting sterol particles of very low organic solvent-content, which are suitable for direct use including oral intake by humans. Also, clandering, pressing, melting and spray-(dry)-ing are suitable particle forming processes.

In an embodiment of the presently claimed invention, for measurement of the Gardner color number, the phytosterol is provided in the form of a 10% by weight solution in pyridine.

In an embodiment of the presently claimed invention, the final sterol product has a Gardner color number of less than 4.0, when measured for a 10 wt. % of sterol in pyridine.

In an embodiment of the presently claimed invention, the final sterol product has a Gardner color number of less than 3.0, preferably less than 2.0, more preferably less than 1.5, even more preferably less than 1, such as less than 0.9, 0.8, 0.7, 0.6, 0.5 and any value in between and below 4 and 0.5, when measured for a 10 wt. % of the sterol in pyridine.

In an embodiment of the presently claimed invention, the solvent content in the purified phytosterol is less than 100 ppm, preferably less than 50 ppm, more preferably less than 20 ppm, and even more preferably less than 10 ppm such as 5 or 1 ppm or even below, and every value in between 100 and 1 ppm, based on the total weight of the purified phytosterol.

In an embodiment of the presently claimed invention, the sterol ester content in the purified phytosterol is less than 10% by weight, preferably less than 5% by weight, more preferably less than 2% by weight, even more preferably less than 1% by weight, and most preferably less than 0.5% by weight, such as 0.1, 0.05% by weight and every value in between 5 and 0.05% by weight and below, based on the total weight of the purified phytosterol.

Advantages

The presently claimed invention is associated with at least one, preferably two and even more preferably all three of the following advantages:

1. The phytosterols are obtained with a Gardner color number of less than 4.0, preferably less than 3.0, preferably less than 2.0, more preferably less than 1.5, even more preferably less than 1, such as less than 0.9, 0.8, 0.7, 0.6, 0.5 and any value in between and below 4 and 0.5, when measured for a 10 wt. % of the sterol in pyridine

2. The phytosterols are obtained in a high yield with a very low content of sterol ester (i.e. <10%) by using the above described purification process, preferably less than 5% by weight, more preferably less than 2% by weight, even more preferably less than 1% by weight, and most preferably less than 0.5% by weight, such as 0.1, 0.05% by weight and every value in between 5 and 0.05% by weight and below, based on the total weight of the purified phytosterol.

3. The solvent content of the final product is low, i.e. less than 100 ppm, preferably less than 50 ppm, more preferably less than 10 ppm such as 5 or 1 ppm or even below, and every value in between 100 and 1 ppm, based on the total weight of the purified phytosterol.

In the following, there is provided a list of embodiments to further illustrate the present disclosure without intending to limit the disclosure to the specific embodiments listed below.

### Preferred Embodiments

1. A process for purification of phytosterols, said process comprising at least the steps of:
   a. providing a liquid mixture comprising a phytosterol and a lower alcohol, wherein the lower alcohol is present in an amount of from 25 to 800% by weight, based on the amount of phytosterol;
   b. cooling the liquid mixture of step a. to form phytosterol crystals, wherein the phytosterol crystals are formed at a temperature of from 10° C. to 75° C., preferably 15° C. to 50° C., more preferably 20° C. to 45° C., even more preferably 25° C. to 35° C.;
   c. separating the phytosterol crystals of step b. from the remainder of the liquid mixture by mechanical means such as filtration and/or centrifugation;
   d. subjecting the phytosterol crystals of step c. to washing with a solvent system comprising at least one polar aprotic solvent to obtain purified phytosterol crystals;
   e. optionally repeating step (d) once, twice or three times, preferably once;
   f. optionally subjecting the phytosterol crystals obtained in step d or—if employed—step e to a washing using at least one lower alcohol, preferably only methanol, and optionally repeat this step f once or twice or three time, preferably only once or twice, more preferably only once,
   g. drying the purified phytosterol crystals;
   h. optionally melting and drying in molted state to remove traces of remaining solvent;
   i. optionally subjecting to a particle-forming process to obtain solid sterol particles
2. A process for purification of phytosterols comprises at least the steps of,
   a. providing a liquid mixture comprising a phytosterol and a lower alcohol, wherein the lower alcohol is present in an amount of from 25 to 800% by 50 weight, based on the amount of phytosterol;
   b. cooling the liquid mixture of step a. to form phytosterol crystals, wherein the phytosterol crystals are formed at a temperature of from 15° C. to 45° C., preferably 20° C. to 30° C.;
   c. separating the phytosterol crystals of step b. from the remainder of the liquid 55 mixture by mechanical means such as filtration and/or centrifugation;
   d. washing the phytosterols crystals of step c. with a solvent system comprising at least one polar aprotic solvent and at least one polar protic solvent to obtain purified phytosterol crystals;
   e. optionally repeating step d. at least once, more preferably at least 2 times, even more preferably at least 3 times;
   f. optionally subjecting the phytosterol crystals obtained in step d or—if employed—step e to a washing using at least one lower alcohol, preferably only methanol, and optionally repeat this step f once or twice or three time, preferably only once or twice, more preferably only once,
   g. optionally repeating steps a. to e. by re-dissolving the phytosterols in a liquid mixture according to step a. and then repeating steps b. to f.;
   h. drying the purified phytosterol crystals obtained from the preceding step;
   i. optionally melting and drying in molted state to remove traces of remaining solvent;
   j. optionally subjecting to a particle-forming process to obtain solid sterol particles.
3. The process according to embodiment 1 or 2, wherein with the at least one polar aprotic solvent being selected from the group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, with ethyl acetate, acetone, methyl ethyl ketone, methyl acetate, heptane and hexane being preferred, and with ethyl acetate, methyl ethyl ketone and methyl acetate being even more preferred, and with the at least one polar aprotic solvent being selected from the group of water, ethanol, methanol, isopropyl alcohol, butanol and acetic acid, with water, ethanol, methanol and/or isopropyl alcohol being more preferred and methanol being most preferred, even more preferably methanol as sole polar aprotic solvent.
4. The process according to any of the preceding embodiments, wherein the lower alcohol is present in an amount of from 30 to 50% by weight, based on the amount of phytosterol.
5. The process according to any of the preceding embodiments, wherein the lower alcohol is selected from the group consisting of ethanol, methanol and isopropyl alcohol.
6. The process according to any of the preceding embodiments, wherein the at least one polar aprotic solvent is selected from the group consisting of ethyl acetate, methyl ethyl ketone, acetone and methyl acetate.
7. The process according to any of the preceding embodiments, wherein the solvent system further comprises at least one polar protic solvent.
8. The process according to embodiment 7, wherein the polar protic solvent is selected from the group consisting of water, ethanol, methanol, isopropyl alcohol.
9. The process according to any of the preceding embodiments, wherein the solvent system comprises the polar protic solvent and the at least one polar aprotic solvent as a mixture or an azeotrope.
10. The process according to any of the preceding embodiments, wherein the liquid 50 mixture comprises a phytosterol-containing fraction produced via a process including transesterifications and distillations of vegetable-based oils.
11. The process according to embodiment 10, wherein the oil is selected from the group consisting of rapeseed oil, sunflower oil and soybean oil.
12. The process according to any of the preceding embodiments, further comprising the step of washing the separated phytosterol crystals obtained in step d. with a fatty acid ester.
13. A process for purification of phytosterols, said process comprising at least the steps of:
   a. providing a liquid mixture comprising a phytosterol and a lower alcohol, wherein the lower alcohol is present in an amount of from 25 to 800% by weight, based on the amount of phytosterol;
   b. cooling the liquid mixture of step a. to form phytosterol crystals, wherein the phytosterol crystals are formed at a temperature of from 10° C. to 75° C., preferably 15° C. to 50° C., more preferably 20° C. to 45° C., even more preferably 25° C. to 35° C.;
   c. separating the phytosterol crystals of step b. from the remainder of the liquid mixture by mechanical means such as filtration and/or centrifugation;
   d. washing the phytosterols crystals of step c. with a solvent system comprising at least one polar aprotic solvent and at least one polar protic solvent to obtain purified phytosterol crystals;
   e. optionally repeating the steps a. to d.;
   f. optionally subjecting the phytosterol crystals obtained in step d or—if employed—step e to a washing using at least one lower alcohol, preferably only methanol, and optionally repeat this step f once or twice or three time, preferably only once or twice, more preferably only once,
   g. optionally repeating step e. by re-dissolving, the phytosterols to obtain a liquid mixture according to step a. and then repeating steps b. to f.;
   h. drying the purified phytosterol crystals;
   i. optionally melting and drying in molted state to remove traces of remaining solvent;
   j. optionally subjecting to a particle-forming process to obtain solid sterol particles.
14. A process for purification of phytosterols, said process comprising,
   a. providing a phytosterol-containing fraction produced via transesterification of an oil selected from the group consisting of rapeseed oil, sunflower oil and soybean oil, wherein the phytosterol-containing fraction comprises a phytosterol and methanol, wherein the methanol is present in an amount of from 30 to 50% by weight, based on the amount of phytosterol;
   b. cooling the phytosterol-containing fraction of step a. to form phytosterol crystals, wherein the phytosterol crystals are formed at a temperature of from 22° C. to 35° C.;
   c. separating the phytosterol crystals of step b. from the remainder of the phytosterol-containing fraction by mechanical means such as filtration and/or centrifugation;
   d. subjecting the phytosterol crystals of step c. to washing with a solvent system comprising at least one polar aprotic solvent and at least one polar protic solvent to obtain purified phytosterol crystals;
   e. optionally repeating step d. once, twice or three times;
   f. optionally subjecting the phytosterol crystals obtained in step d or—if employed—step e to a washing using at least one lower alcohol, preferably only methanol, and optionally repeat this step f once or twice or three time, preferably only once or twice, more preferably only once;
   g. drying the purified phytosterol crystals;
   h. optionally melting and drying in molted state to remove traces of remaining 50 solvent;
   i. optionally subjecting to a particle-forming process to obtain solid sterol particles.
15. The process according to embodiment 13 or 14, wherein with the at least one polar 55 aprotic solvent being selected from the group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, with ethyl acetate, acetone, methyl ethyl ketone, methyl acetate, heptane and hexane being preferred, and with ethyl acetate, methyl ethyl ketone and methyl acetate being even more preferred, and with the at least one polar aprotic solvent being selected from the group of water, ethanol, methanol, isopropyl alcohol, butanol and acetic acid, with water, ethanol, methanol and/or isopropyl alcohol being more preferred and methanol being most preferred, even more preferably methanol as sole polar aprotic solvent.
16. The process according to any of the embodiments 13 to 15, wherein the at least one polar aprotic solvent is selected from the group consisting of ethyl acetate, acetone, methyl ethyl ketone and methyl acetate.
17. The process according to any of the embodiments 13 or 16, wherein the at least one polar protic solvent is selected from the group consisting of water, ethanol, methanol and isopropyl alcohol.
18. The process according to any of the embodiments 13 to 16, wherein the solvent system comprises the at least one polar protic solvent and the at least one polar aprotic solvent as a mixture or an azeotrope.

### EXAMPLES

The presently claimed invention is illustrated in detail by non-restrictive working examples which follow. More particularly, the test methods specified hereinafter are part of the general disclosure of the application and are not restricted to the specific working examples.

The starting material, the “residue”, used for Example 1 was obtained using the process as disclosed in EP1179535B1, using rape seed oil as starting material for the process. The residue as obtainable from the processes as disclosed in EP1179536B1 and EP 2 635 592 B1 yield a very similar product to that of EP1179535B1, and thus are equally suitable as well and thus 30 can be submitted to the very same process further outlined here in the present examples 1 and 2.

### Example 1

The starting material used was a rapeseed methyl ester residue which, based on the content of free and bound sterols, additionally contained 100% by weight of methanol.

The mixture was continuously cooled from ca. 100° C. to 10° C., the first crystals beginning to separate at 65° C. The reaction mixture is cooled from 65° C. to about 25-30° C. On completion of the crystallization, the crystals were filtered off, washed free from FME with methanol and 40 dried to constant weight. The yield was 78% by weight, based on the sterol content of the transesterification product.

### Example 2—Purification of the Sterol Crystals

On completion of the crystallization as performed according to Example 1, the crystals were filtered off, washed free from FME with pure methanol and further subjected to washings with the following solvents:
ethyl acetate and its azeotrope with methanol, or
methyl ethyl ketone and its azeotrope with methanol, or
methyl acetate and its azeotrope with methanol,

with subsequent pure methanol washing.

Further the crystals were melt dried by known methods to constant weight and subjected to particle forming by prilling.

The results obtained were compared to experiments, in which the crystals were washed with FME with subsequent pure methanol washing.

The results obtained are summarized in Tables 1-3 (laboratory scale) as well as in Tables 1a-3a (commercial scale, e.g. plant level). In each table, example C1 and T1 refer to the same sterol batch originating from the same rapeseed methyl ester distillation residue. The same applies to C2 and 12, 3 and 13 as well as C4 and T4 (if applicable). Thus, example C1 has to be compared with example T1 and so on.

TABLE 1
Solvent used is azeotrope of ethyl acetate
with methanol (laboratory scale)

| Expt. | | | | |
|---|---|---|---|---|
| No. | Solvent for washing | Color | Purity | Yield |
| C1 | 1 washing of FME + 3 washings of | 2.1 | 98.8 | 73.7 |
| | methanol | | | |
| C2 | 1 washing of FME + 3 washings of | 2.4 | 96.8 | 65.6 |
| | methanol | | | |
| C3 | 1 washing of FME + 3 washings of | 4 | 98.4 | 58.1 |
| | methanol | | | |
| C4 | 1 washing of FME + 3 washings of | 1 | 99.6 | 72 |
| | methanol | | | |
| T1 | 2 washings of azeotrope of ethyl acetate/ | 1.2 | 99.5 | 74.6 |
| | methanol + 1 washing of methanol | | | |
| T2 | 2 washings of azeotrope of ethyl acetate/ | 1.2 | 98.4 | 72.6 |
| | methanol + 1 washing of methanol | | | |
| T3 | 2 washings of azeotrope of ethyl acetate/ | 3 | 100 | 69.5 |
| | methanol + 1 washing of methanol | | | |
| T4 | 2 washings of azeotrope of ethyl acetate/ | 0.6 | 99.7 | 72 |
| | methanol + 1 washing of methanol | | | |

TABLE 1a
Solvent used is azeotrope of ethyl acetate
with methanol (commercial scale)

| Expt. | | | | |
|---|---|---|---|---|
| No. | Solvent for washing | Color | Purity | Yield |
| C1 | 1 washing of FME + 3 washings of | 2.7 | n.d. | 62.5 |
| | methanol | | | |
| T1 | 2 washings of azeotrope of ethyl | 0.7 | n.d. | 61.2 |
| | acetate/methanol + 1 washing of | | | |
| | methanol | | | |

n.d. = not determined

TABLE 2
Solvent used is azeotrope of methyl ethyl
ketone with methanol (laboratory scale)

| Expt. | | | | |
|---|---|---|---|---|
| No. | Solvent for washing | Color | Purity | Yield |
| C1 | 1 washing of FME + 3 washings of | 3.6 | 99.2 | 54.4 |
| | methanol | | | |
| C2 | 1 washing of FME + 3 washings of | 2.7 | 99.1 | 62.0 |
| | methanol | | | |
| C3 | 1 washing of FME + 3 washings of | 4 | 98.4 | 58.1 |
| | methanol. | | | |
| T1 | 2 washings of azeotrope of methyl ethyl | 1.9 | 99.3 | 70.3 |
| | ketone/methanol + 1 washing of | | | |
| | methanol | | | |
| T2 | 2 washings of azeotrope of methyl ethyl | 1.9 | 99.4 | 75.7 |
| | ketone/methanol + 1 washing of | | | |
| | methanol | | | |
| T3 | 2 washings of azeotrope of methyl ethyl | 2.8 | 99.4 | 70.6 |
| | ketone/methanol + 1 washing of | | | |
| | methanol | | | |

TABLE 2a
Solvent used is azeotrope of methyl ethyl
ketone with methanol (commercial scale)

| Expt. | | | | |
|---|---|---|---|---|
| No. | Solvent for washing | Color | Purity | Yield |
| C1 | 1 washing of FME + 3 | 1.9 | n.d. | 59.2 |
| | washings of methanol | | | |
| T1 | 2 washings of azeotrope | 1.2 | n.d. | 64.9 |
| | of methyl ethyl ketone/ | | | |
| | methanol + 1 washing of | | | |
| | methanol | | | |

n.d. = not determined

TABLE 3
Solvent used is azeotrope of methyl acetate
with methanol (laboratory scale)

| Expt. | | | | |
|---|---|---|---|---|
| No. | Solvent for washing | Color | Purity | Yield |
| C1 | 1 washing of FMEFME + 3 washings | 4 | 98.4 | 58.4 |
| | of methanol | | | |
| C2 | 1 washing of FME + 3 washings of | 2.7 | 99.1 | 62.6 |
| | methanol | | | |
| C3 | 1 washing of FME + 3 washings of | 3.6 | 99.1 | 62.6 |
| | methanol | | | |
| C4 | 1 washing of FME + 3 washings of | 3.0 | 100 | 75.1 |
| | methanol | | | |
| T1 | 2 washing of azeotrope of methyl | 3.2 | 99.3 | 71.8 |
| | acetate/methanol + 1 washing of | | | |
| | methanol | | | |
| T2 | 2 washing of azeotrope of methyl | 1.2 | 99.1 | 74.7 |
| | acetate/methanol + 1 washing of | | | |
| | methanol | | | |
| T3 | 2 washing of azeotrope of methyl | 1.6 | 99.1 | 74.7 |
| | acetate/methanol + 1 washing of | | | |
| | methanol | | | |
| T4 | 3 washing of azeotrope of methyl | 0.5 | 100 | 77 |
| | acetate/methanol | | | |

TABLE 3a
Solvent used is azeotrope of methyl acetate
with methanol (commercial scale)

| Expt. | | | | |
|---|---|---|---|---|
| No. | Solvent for washing | Color | Purity | Yield |
| C1 | 1 washing of FME + 3 washings of | 2.2 | n.d. | 62.7 |
| | methanol | | | |
| T1 | 2 washings of azeotrope of methyl | 1.0 | n.d. | 67.7 |
| | acetate/methanol + 1 washing of | | | |
| | methanol | | | |

n.d. = not determined

### CONCLUSIONS

According to Table 1 (laboratory scale), the color of the final sterol product is remarkedly better in case of T1 to T4 than in case of C1 to C4. In comparison to the latter, the purity is at least slightly better, whereas, the yield is at least the same or even better for T1 to T4. As can be taken from Table 1a (plant level), the color is strongly improved for T1 compared to C1, whereas, the yield more or less stays the same.

According to Table 2 (laboratory scale), the color and especially the yield of the final sterol product is remarkedly better in case of T1 to T3 than in case of C1 to C3. In comparison to the latter, the purity is slightly better for T1 to T3. As can be taken from Table 2a (plant level), color and yield are improved for T1 compared to C1.

According to Table 3 (laboratory scale), the yield and especially the color of the final sterol product is remarkedly better in case of T1 to T4 than in case of C1 to C4. In comparison to the latter, the purity is at least the same or even slightly better for T1 to T4. As can be taken from Table 3a (plant level), color and yield are improved for T1 compared to C1.

It will be appreciated by those skilled in the art that changes could be made to the embodiments described above without departing from the broad inventive concept thereof.

It is understood, therefore, that this invention is not limited to the particular embodiments disclosed, but it is intended to cover modifications within the spirit and scope of the presently claimed invention as defined by the appended claims.

1.-19. (canceled)

20. A process for purification of phytosterols, said process comprising at least the steps of:
a. providing a liquid mixture comprising a phytosterol and a lower alcohol, wherein the lower alcohol is present in an amount of from 25 to 800% by weight, based on the amount of phytosterol;
b. cooling the liquid mixture of step a. to form phytosterol crystals, wherein the phytosterol crystals are formed at a temperature of from 10° C. to 75° C.;
c. separating the phytosterol crystals of step b. from the remainder of the liquid mixture by mechanical means such as filtration and/or centrifugation;
d. subjecting the phytosterol crystals of step c. to washing with a solvent system comprising at least one polar aprotic solvent to obtain purified phytosterol crystals;
e. optionally repeating step (d) once, twice or three times;
f. optionally subjecting the phytosterol crystals obtained in step d or—if employed—step e to a washing using at least one lower alcohol, and optionally repeat this step f once or twice or three time;
g. drying the purified phytosterol crystals;
h. optionally melting and drying in molted state to remove traces of remaining solvent;
i. optionally subjecting to a particle-forming process to obtain solid sterol particles.

21. A process for purification of phytosterols comprises at least the steps of,
a. providing a liquid mixture comprising a phytosterol and a lower alcohol, wherein the lower alcohol is present in an amount of from 25 to 800% by weight, based on the amount of phytosterol;
b. cooling the liquid mixture of step a. to form phytosterol crystals, wherein the phytosterol crystals are formed at a temperature of from 15° C. to 45° C.;
c. separating the phytosterol crystals of step b. from the remainder of the liquid mixture by mechanical means such as filtration and/or centrifugation;
d. washing the phytosterols crystals of step c. with a solvent system comprising at least one polar aprotic solvent and at least one polar protic solvent to obtain purified phytosterol crystals;
e. optionally repeating step d. at least once,
f. optionally subjecting the phytosterol crystals obtained in step d or—if employed step e to a washing using at least one lower alcohol,
g. optionally repeating steps a. to f. by re-dissolving the phytosterols in a liquid mixture according to step a. and then repeating steps b. to f.;
h. drying the purified phytosterol crystals obtained from the preceding step;
i. optionally melting and drying in molted state to remove traces of remaining solvent;
j. optionally subjecting to a particle-forming process to obtain solid sterol particles.

22. The process according to claim 20, wherein the at least one polar aprotic solvent is selected from the group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, with ethyl acetate, acetone, methyl ethyl ketone, methyl acetate, heptane, and
wherein the at least one polar aprotic solvent is selected from the group of water, ethanol, methanol, isopropyl alcohol, butanol and acetic acid.

23. The process according to claim 20, wherein the lower alcohol is present in an amount of from 30 to 50% by weight, based on the amount of phytosterol.

24. The process according to claim 20, wherein the lower alcohol is selected from the group consisting of ethanol, methanol and isopropyl alcohol.

25. The process according to claim 20, wherein the at least one polar aprotic solvent is selected from the group consisting of ethyl acetate, acetone, methyl ethyl ketone and methyl acetate.

26. The process according to claim 20, wherein the solvent system further comprises at least one polar protic solvent.

27. The process according to claim 26 wherein the polar protic solvent is selected from the group consisting of water, ethanol, methanol, isopropyl alcohol.

28. The process according to claim 20, wherein the solvent system comprises the polar protic solvent and the at least one polar aprotic solvent as a mixture or an azeotrope.

29. The process according to claim 20, wherein the liquid mixture comprises a phytosterol-containing fraction produced via a process including transesterifications and distillations of vegetable-based oils.

30. The process according to claim 29, wherein the oil is selected from the group consisting of rapeseed oil, sunflower oil and soybean oil.

31. The process according to claim 20, further comprising the step of washing the separated phytosterol crystals obtained in step d. with a fatty acid ester.

32. A process for purification of phytosterols, said process comprising at least the steps of:
a. providing a liquid mixture comprising a phytosterol and a lower alcohol, wherein the lower alcohol is present in an amount of from 25 to 800% by weight, based on the amount of phytosterol;
b. cooling the liquid mixture of step a. to form phytosterol crystals, wherein the phytosterol crystals are formed at a temperature of from 10° C. to 75° C.;
c. separating the phytosterol crystals of step b. from the remainder of the liquid mixture by mechanical means such as filtration and/or centrifugation;
d. washing the phytosterols crystals of step c. with a solvent system comprising at least one polar aprotic solvent and at least one polar protic solvent to obtain purified phytosterol crystals;
e. optionally repeating the steps a. to d.;
f. optionally repeating step e. by re-dissolving, the phytosterols to obtain a liquid mixture according to step a. and then repeating steps b. to f.;
g. drying the purified phytosterol crystals;
h. optionally melting and drying in molted state to remove traces of remaining solvent;
i. optionally subjecting to a particle-forming process to obtain solid sterol particles.

33. A process for purification of phytosterols, said process comprising,
a. providing a phytosterol-containing fraction produced via transesterification of an oil selected from the group consisting of rapeseed oil, sunflower oil and soybean oil, wherein the phytosterol-containing fraction comprises a phytosterol and methanol, wherein the methanol is present in an amount of from 30 to 50% by weight, based on the amount of phytosterol;
b. cooling the phytosterol-containing fraction of step a. to form phytosterol crystals, wherein the phytosterol crystals are formed at a temperature of from 22° C. to 35° C.;
c. separating the phytosterol crystals of step b. from the remainder of the phytosterol-containing fraction by mechanical means such as filtration and/or centrifugation;
d. subjecting the phytosterol crystals of step c. to washing with a solvent system comprising at least one polar aprotic solvent and at least one polar protic solvent to obtain purified phytosterol crystals;
e. optionally repeating step d.;
f. drying the purified phytosterol crystals;
g. optionally melting and drying in molted state to remove traces of remaining solvent;
h. optionally subjecting to a particle-forming process to obtain solid sterol particles.

34. The process according to claim 32, wherein the at least one polar aprotic solvent is selected from the group consisting of ethyl acetate, methyl ethyl ketone, methyl acetate, dichloromethane, N-methyl pyrrolidone, tetrahydrofuran, acetone, dimethylformamide, acetonitrile, dimethyl sulfoxide, heptane, and hexane, and
wherein the at least one polar protic solvent is selected from the group of water, ethanol, methanol, isopropyl alcohol, butanol and acetic acid.

35. The process according to claim 32, wherein the at least one polar aprotic solvent is selected from the group consisting of ethyl acetate, methyl ethyl ketone and methyl acetate.

36. The process according to claim 32, wherein the at least one polar protic solvent is selected from the group consisting of water, ethanol, methanol and isopropyl alcohol.

37. The process according to claim 32, wherein the solvent system comprises the at least one polar protic solvent and the at least one polar aprotic solvent as a mixture or an azeotrope.

38. The process according to claim 20, wherein the ratio of polar aprotic solvent to polar protic solvent in the solvent system is such as to form an azeotropic mixture, and is in the range of 90:10 to 70:30 volume by volume.
